(19) United States
(12) Patent Application Publication
(10) Pub. No.: US 2009/0023211 A1
(43) Pub. Date: Jan. 22, 2009

(54) Blood platelet lysate and method of producing the same

(75) Inventors: Anna Persson, Sodra Sandby (SE); Nicklas Alfredsson, Kavlinge (SE); Kerstin Christensson, Landskrona (SE); Sten Ohlson, Malmo (SE); Olov Holmqvist, Lund (SE)

Correspondence Address: BUCHANAN, INGERSOLL & ROONEY PC, POST OFFICE BOX 1404, ALEXANDRIA, VA, 22313-1404, US

(73) Assignee: PROLIFF AB, KAVLINGE (SE)

(21) Appl. No.: 11/922,412

(22) PCT Filed: Jun. 16, 2006
(86) PCT No.: PCT/SE2006/000720

§ 371 Date: Jan. 25, 2008

(30) Foreign Application Priority Data
Jun. 23, 2005 (SE) 0501462-6

(51) Int. Cl. C12N 5/02; C12N 5/06; A23L 1/31

(52) U.S. Cl. 435/408; 210/650; 426/647

(57) ABSTRACT

A method of preparing a blood platelet lysate starting from platelet-rich plasma from whole blood of animals, to which blood a citrate has been added to prevent coagulation, is described. The method is characterised by concentrating the platelet-rich plasma by ultrafiltration to obtain a plasma that is richer in platelets, adding water for lysis of the platelets included, adding Ca²⁺ to the lysed concentrated platelet-rich plasma for forming coagel of other components than lysed platelets, centrifuging the coagel, whereby a clear, bright-red liquid of blood platelet lysate is obtained, which substantially consists of lysed platelets, and a coagulate, and, after centrifugation, the liquid going through a filtering step.

### FIELD OF THE INVENTION

The present invention relates to a blood platelet lysate obtained from platelet-rich plasma from whole blood of animals, and a method of preparing the blood platelet lysate.

### BACKGROUND ART

A problem in the slaughterhouse industry, in an international perspective, is, inter alia, to find a continuous and profitable market for slaughter blood. In many countries, the blood is even discarded as waste material for lack of possibilities of using the blood. In cases where the slaughter blood is taken care of, it is mainly used as an admixture to foodstuffs and animal feed. The hemoglobin part of the blood can be used for different blood products, such as black-pudding, and the plasma part can be used as a thickener, a protein-enriching agent etc.

European Application published under No. 413,727 discloses use of the slaughter blood in a method of preparing blood platelet lysate, and a culture medium containing the blood platelet lysate. A problem with prior-art methods of preparing blood platelet lysate is, among other things, that the yields obtained are low and that the product does not have all the properties that are required from a cell culture medium.

Another problem in the increasing research where animal cells are used is that one of the main components in culture media that are used for cell culture is foetal bovine serum (FBS) or foetal calf serum (FCS). Foetal bovine serum is extracted from calf foetus from slaughtered cows, and the supply of foetuses has become a limiting factor when preparing these culture media. Also ethical requirements have increased the demand for culture media which have not been prepared by cardiac puncture in live calf foetuses.

In addition, with the attention focused on BSE, it may be advantageous from the viewpoint of exposure hazard to have an origin other than bovine. It has therefore become convenient to prepare culture media having the same functionality as FBS, but from a different kind of animal.

### SUMMARY OF THE INVENTION

An object of the present invention is to provide a concentrated blood platelet lysate, which is obtained from platelet-rich plasma from whole blood of animals, both young and adult animals.

Another object of the present invention is to provide a method of preparing the blood platelet lysate, which method solves the above problems and gives a better product with a higher yield.

According to the present invention, a method is provided for preparing a blood platelet lysate starting from platelet-rich plasma from whole blood of animals, to which whole blood a citrate has been added to prevent coagulation, characterised by
a) concentrating the platelet-rich plasma by ultra-filtration to obtain a concentrated platelet-rich plasma,
b) adding water to the concentrated platelet-rich plasma for lysis of the platelets included,
c) adding Ca²⁺ to the lysed concentrated platelet-rich plasma for forming coagel of other components than lysed platelets, the concentrated platelet-rich plasma, after addition of Ca²⁺ being allowed to stand at 37° C. maximum, preferably 18-25° C., for 3-8 h, preferably about 5 h,
d) centrifuging the coagel, whereby a blood platelet lysate is obtained, which substantially consists of lysed platelets, and a coagulate, and
e) after centrifugation, the blood platelet lysate going through a filtering step.

Furthermore use of a blood platelet lysate is provided, which has been prepared from platelet-rich plasma from whole blood of animals, in a cell culture medium, which comprises, in addition to the blood platelet lysate according to the invention, a conventional nutrient medium and optionally foetal bovine serum or some other factor, for instance fibronectin, which can promote certain cells, for culturing animal cells, such as Vero-, Hybridoma 39.5- and CHO cells.

Preferred embodiments according to the invention are defined in the dependent claims.

The animals intended with the present invention can basically be all animals, both young ones and adults, from which it possible to obtain the required amount of blood. Preferably blood extracted in slaughter is used, and the blood comes mainly from adult animals. Examples of animals that are convenient for use in the present invention are slaughter animals and other farm animals of the type cattle, pig, sheep, goat, horse or poultry. Preferably whole blood of cattle or pig is used.

The animals used in the present invention should be healthy animals which satisfy the requirements that are placed in veterinary inspection of animals intended for food purposes.

The blood platelet lysate obtained according to the present invention is particularly useful for culture of animal cells. The blood platelet lysate can wholly or partly replace foetal bovine serum in cell culture.

### DESCRIPTION OF PREFERRED EMBODIMENTS

After drawing off, the blood of animals is cooled rapidly, preferably to about +4° C., within a minute. A citrate, such as sodium citrate, is added to the blood to prevent coagulation. Optionally, the blood can then be stored in cooling tanks until it is time for further treatment.

Before further treatment, the blood is examined, both in bacteriological and in sensory respect, to ensure that the blood satisfies the requirements which the National Food Administration places on products intended for foodstuffs, and corresponding regulations within the EU and the US. From experience, we have found that suitable upper limits for the bacterial content in blood are those stated in Table 1 below. A sensory analysis is carried out by testing the smell of the blood, where blood having a deviating smell is rejected.

Bacteriological samples are preferably taken from whole blood.

TABLE 1
Bacteriological Limit of Acceptance of the Blood for Use

| | Number of bacteria/g |
|---|---|
| Aerobic number of bacteria | <100 000 |
| (tryptonglucose extract agar | |
| 30° C. for 3 days) | |
| E. coli | <10 |
| Staphylococcus aureus | <100 |

The blood should preferably also have a temperature between 0° C. and 7° C. and be at most 72 h of age.

A separate sample is taken from the whole blood for hemolysis assessment of the plasma part. The hemolysis figure is a measure of the amount of lysed red blood corpuscles and is assessed according to a scale between 1 and 8. The blood used in the present invention preferably has a hemolysis figure of 1-5, more preferred 1-3, still more preferred 1-2.

Based on the results obtained in the analyses, it is determined whether the blood can be used or not.

After analysis, the blood is separated into red blood corpuscles and platelet-rich plasma. Use is preferably made of a blood separator, but also other suitable, traditional separators can be used. All the time the blood is kept cool, suitably at about +4° C.

Subsequently the platelet-rich plasma is concentrated by ultrafiltration, where the protein content of the platelet-rich plasma is concentrated from about 6% to about 20%. After ultrafiltration, a citrate can be added to compensate for the amount of citrate that is lost in concentration, after which the concentrated platelet-rich plasma is optionally frozen for storage and/or analysis and further lysis of the platelets included. After optional freezing, the frozen plasma is thawed, preferably at a temperature below 37° C., more preferred below 20° C., most preferred below 12° C. Thawing occurs due to the temperature restriction advantageously in a room with a controlled temperature, such as a refrigerator or a refrigerating room.

After thawing of the concentrated platelet-rich plasma, water is added for lysis of the platelets included. Water can also be added to the frozen concentrated platelet-rich plasma to accelerate thawing. The water added preferably consists of deionised water, which results in a greater difference in osmotic pressure. The amount of water added preferably is about 1 part water to 2 parts concentrated platelet-rich plasma. Lysis should occur at a temperature which is below 37° C., preferably below 22° C., more preferred below 10° C., still more preferred below 5° C. Lysis preferably occurs with water for at least 1 h.

To obtain a coagel of other components than lysed platelets, a liquid is then added, containing Ca²⁺ ions, for instance in the form of calcium chloride-2-hydrate, preferably at a ratio of 1:1 (concentrated platelet-rich plasma:Ca²⁺ solution).

The mixture is then allowed to stand at 37° C. maximum, preferably about 18-25° C., for 3-8 h, preferably for about 5 h. After about 3 h, it should be checked that the coagulation has started. If there is no coagulation, or only poor coagulation, further Ca²⁺ ions are added. Coagulation at room temperature (about 20° C.) is important. If coagulation occurs at a lower temperature, or alternatively for a shorter period of time, there is a great risk that the mixture has not coagulated completely. In this case, post-coagulation can occur in the filtration of the blood platelet lysate, or alternatively when the filtered blood platelet lysate is stored frozen or is used at room temperature. In coagulation for an excessive time, optionally in combination with an excessive temperature, microbiological problems may arise due to microbiological growth.

After coagulation, the coagel formed is decomposed and then centrifuged, resulting in a clear, bright-red supernatant, blood platelet lysate, which contains the major part of the lysed platelets, the concentrated coagel being obtained as bottom settlings. In a preferred embodiment, the coagel is centrifuged at about 6000 RCF (Relative Centrifugal Force) for 30 min and at about 20° C.

Also other concentration steps which are to be compared to centrifugation and filtering, such as those based on the affinity principle, can be used in the method according to the present invention.

After centrifugation, the supernatant, that is the blood platelet lysate, is poured off and stored. In a preferred embodiment, citrate is then added to the blood platelet lysate, preferably 0.1-2.0 weight %, more preferred 0.3-1.0 weight %, to bind any excess of Ca²⁺ ions and thus reduce the risk of a possible post-coagulation. After centrifugation, the blood platelet lysate goes through a filtering step. The filtering step preferably comprises filtration through a screen/filter cloth and/or sterile filtration. When the filtering step comprises filtration through a screen/filter cloth, this can be performed immediately after centrifugation, but also after an optional addition of citrate. In a preferred embodiment of the present invention, the filtering step also comprises prefiltration before the blood platelet lysate is sterile-filtered.

The method according to the invention can occur continuously, batchwise or as a combination of both. If desired, also further concentration steps, such as centrifugation step, filtering step etc, can be introduced. The method can also be optimised, for instance, by additional centrifugation and filtering, to increase the yield and the quality of the final product.

The cell culture medium that can be obtained in use of the blood platelet lysate according to the present invention comprises, in addition to the present blood platelet lysate, a nutrient medium containing salts, factor, etc. and optionally FBS. The nutrient medium can be any suitable, conventional nutrient medium, for instance one stated in the Sigma catalogue issued by Sigma Chemical Company.

The invention will now be further illustrated with reference to the following Examples. The Examples are only intended to be elucidative and should in no way be considered to restrict the invention.

### EXAMPLES

### Example 1a

### Preparation of 5 Batches of Bovine Blood Platelet Lysate

The platelet-rich plasma was taken from a continuous process for separating platelet-rich plasma from whole blood from slaughter cattle. A sensory and bacteriological analysis of the whole blood gave results that were acceptable according to that described above. The plasma in the whole blood had a hemolysis figure varying between 1 and 4. About 60% platelet-rich plasma was obtained from the whole blood while using a blood separator of the Alfa Laval type HMRPX 714 HGV, that is about 3000 l platelet-rich plasma was obtained from 5000 l blood.

The platelet-rich plasma was concentrated to a protein content of about 12% by ultrafiltration while using an X-Flow membrane of the tubular type of polysulphone with a cut-off of 100000 Dalton. About 1500 l concentrated platelet-rich plasma was obtained. To the concentrated platelet-rich plasma, 16 l sodium citrate solution (25 kg trisodium citrate dissolved in 75 kg water) was added per 1000 l concentrated platelet-rich plasma to compensate for the amount of citrate lost in ultrafiltration. The concentrated platelet-rich plasma was frozen in flakes and analysed with respect to protein, iron and endotoxin content. The citrate content was assumed to be 7.5 g/kg concentrated platelet-rich plasma. Then one part concentrated platelet-rich plasma was taken out of the freezer and thawed, and deionised water was added at a ratio of 1:0.5 (concentrated platelet-rich plasma:water) for lysis of platelets, and the mixture was allowed to stand for at least 1 h. In the meantime, a CaCl₂ solution was mixed, containing 3 g calcium chloride-2-hydrate per 1000 g deionised water.

After lysis, the CaCl₂ solution was added during stirring, at a ratio of 1:1 (concentrated platelet-rich plasma:CaCl₂ solution) and the mixture was allowed to stand at room temperature for about 5 h. Extra Ca²⁺ ions had to be added to three of the five batches to achieve sufficient coagulation.

After coagulation, the coagel was decomposed for centrifugation in a centrifuge of the type Sorvall RC5C, rotor H-12000, at 6000 RCF, 20° C., for 30 min, resulting in a supernatant, blood platelet lysate, and a concentrated coagel. Subsequently the blood platelet lysate was filtered through a screen.

Then sodium citrate (0.375 weight %) was added to the blood platelet lysate. After that the blood platelet lysate was filtered through two Millipore filters; first through a prefilter of the type Milligard CWSSM4S03 and then through a sterile filter of the type Millipak Gamma Gold MPGL2GCA3. The sterile blood platelet lysate was filtered down into sterile E flasks and then transferred to sterile tubes for storage. The tubes were then stored in a freezer at about −18° C.

Table II shows the conditions in which the five batches of bovine blood platelet lysate (BBPL) were prepared.

TABLE II

| | BBPL | BBPL | BBPL | BBPL | BBPL |
|---|---|---|---|---|---|
| Designation | (21) | (22) | (28) | (05) | (10) |
| Thawing (time, | 4 h at | 24 h, | 4 h at | 24 h, | 24 h, |
| temp) | 20° C. + | 4° C. | 20° C. + | 4° C. | 4° C. |
| | 17 h at | | 17 h at | | |
| | 4° C. | | 4° C. | | |
| Amount of conc. | 906 | 800 | 1006 | 862 | 964 |
| platelet-rich | | | | | |
| plasma (g) | | | | | |
| Amount of water | 453 | 400 | 507 | 431 | 482 |
| for lysis (g) | | | | | |
| Time and temp | 1 h, | 1 h at | 1 h, | 1 h at | 12 h |
| for lysis | 20° C. | 4° C. + | 20° C. | 4° C. + | |
| | | 7 days | | 7 days | |
| | | at | | at | |
| | | −18° C. | | −18° C. | |
| Amount of CaCl₂ | 907 | 800 | 1006 | 862 | 964 |
| solution for | | | | | |
| coagulation (g) | | | | | |
| Extra addition | 0 | 2.4 | 3.5 | 0 | 2.8 |
| of CaCl₂ (g) | | | | | |
| Time and temp | 5.5 h, | 5 + 1 h, | 5 + 1 h, | 5 h, | 4 + 1 h, |
| for coagulation | 20° C. | 20° C. | 20° C. | 20° C. | 20° C. |
| Obtained amount | 2112 | 1903 | 2363 | 1977 | 2285 |
| of solution (g) | | | | | |
| Added amount of | 7.9 | 7.1 | 9.3 | 7.4 | 8.7 |
| Na citrate (g) | | | | | |

### Example 1b

### Preparation of Porcine Blood Platelet Lysate

Platelet-rich plasma was taken from a continuous method of separating platelet-rich plasma from whole blood from slaughter pigs. A sensory and bacteriological analysis of the whole blood gave results that were acceptable in accordance with that described above. The plasma in the whole blood had a hemolysis figure varying between 1 and 4. About 55% platelet-rich plasma was obtained from the whole blood, using a blood separator of the type Alfa Laval HMRPX 714 HGV, that is about 2750 l platelet-rich plasma was obtained from 5000 l blood.

The platelet-rich plasma was first concentrated to a protein content of about 12% by ultrafiltration, using an X-Flow membrane of the tubular type of polysulphone with a cut-off of 100,000 Dalton. About 1375 l concentrated platelet-rich plasma was obtained. To the concentrated platelet-rich plasma, there was added 15 l sodium citrate solution (25 kg trisodium citrate dissolved in 75 kg water) per 1000 l concentrated platelet-rich plasma to compensate for the amount of citrate lost in ultrafiltration. The concentrated platelet-rich plasma was frozen in flakes and analysed with respect to protein, iron and endotoxin content.

After that the concentrated platelet-rich plasma was taken out of the freezer and thawed, and deionised water was added at a ratio of 1:0.5 (concentrated platelet-rich plasma:water), and the mixture was allowed to stand for 1 h.

A CaCl₂ solution containing 3 g calcium chloride 2-hydrate per 1000 g deionised water was mixed. After lysis, the CaCl₂ solution was added during stirring at a ratio of 1:1 (concentrated platelet-rich plasma:solution), and the mixture was allowed to stand at room temperature for about 5 h.

After coagulation, the coagel was decomposed for centrifugation in a centrifuge of the type Sorvall RC5C, rotor H-12000, at 6000 RCF, 20° C., for 30 min, which resulted in a supernatant, blood platelet lysate, and a concentrated coagel. Then the blood platelet lysate was filtered through a screen.

Sodium citrate (0.375 weight %) was then added to the blood platelet lysate. Subsequently the blood platelet lysate was filtered through two Millipore filters; first through a prefilter of the type Milligard CWSSM4S03 and then through a sterile filter of the Millipak Gamma Gold MPGL2GCA3. The sterile blood platelet lysate was filtered down into sterile E flasks and then transferred to sterile tubes for storage in a freezer at about −18° C.

Table III shows the conditions in which the porcine blood platelet lysate ((PBPL) was prepared.

TABLE III

| | PBPL (03) |
|---|---|
| Thawing (time, temp) | 5 h at 20° C. + 17 h |
| | at 4° C. |
| Amount of conc. platelet-rich | 991 |
| plasma | |
| (g) | |
| Amount of water for lysis | 503 |
| (g) | |
| Time and temp for lysis | 1 h, 20° C. |
| Amount of CaCl₂₋ solution for | 950 |
| coagulation | |
| (g) | |
| Extra addition of CaCl₂ | 0 |
| (g) | |
| Time and temp for coagulation | 5 h, 20° C. |
| Obtained amount of solution | 1856 |
| (g) | |
| Added amount of Na citrate | 7.0 |
| (g) | |

### Example 2

### Cell Culture Experiment Using Blood Platelet Lysate

The cells were cultured in Dulbecco's Modified Eagle's Medium (DMEM) with a high glucose content (4.5 g/l) with the addition of 0.1% penicillin streptomycin (PEST), 4 mM 1-glutamine and 10% and respectively 5% blood platelet lysate according to the invention or 5% FBS or 5% and respectively 10% commercial PS (porcine serum). 5% FBS and respectively 5% and 10% PS were used for checking. The cells were cultured at 37° C. and 7.5% CO₂. Before seeding, the cells were adapted to the new culture medium during at least two passages, where the cells were divided about 1:5 to 1:10 in T-25 flasks. After this adaptation of the cells, upscaling to T-75 flasks occurred. To determine the concentration of cells that were harvested for seeding, the cells were counted in a Burker chamber and stained with trypan blue. A flow cytometer was used for counting the start concentration of cells after seeding and the concentration on each cell counting day. The flow cytometer used was of the type FACSCalibur Becton Dickinson Immunocytometry Systems, San Jose, USA.

The viability of the cells was measured by fluorescence staining of the type Sytox Green Nucleic Acid Stain (Molecular Probes, Eugene, USA). Sytox penetrates intact cell membranes. This take-up is seen as green fluorescence in excitation from an argon laser beam at 488 nm.

The cell lines used were of the type Vero (African green monkey transformed kidney epithelial cells), Hybridoma 39.5 derived from mice and CHO cells (Chinese Hamster Ovary).

The cell concentration and viability were measured on the flow cytometer. To analyse the efficiency of the blood platelet lysate according to the present invention, a ratio of the cell concentration in each measurement to the seeding concentration was calculated. With this calculation, each seeding concentration was taken into consideration.

### Example 2a

### Cell Culture Experiment Using Bovine Blood Platelet Lysate

Table IV shows the ratio of the cell concentration for selected days to the seeding concentration for Vero cells. The experiment was performed with bovine blood platelet lysate (BBPL), 5 different batches, and with FBS as a reference.

TABLE IV

| | | 10% BBPL | 10% BBPL | 10% BBPL | 10% BBPL | 10% BBPL |
|---|---|---|---|---|---|---|
| Day | 5% FBS | (05) | (10) | (21) | (22) | (28) |
| 0 | 1.00 | 1.00 | 1.00 | 1.00 | 1.00 | 1.00 |
| 1 | 0.51 | 0.47 | 0.53 | 0.41 | 0.51 | 0.37 |
| 4 | 9.64 | 9.77 | 10.01 | 6.83 | 8.17 | 9.10 |
| 7 | 19.79 | 15.66 | 11.37 | 14.09 | 10.15 | 13.21 |
| 8 | 19.00 | 11.31 | 8.77 | 9.13 | 5.65 | 10.19 |

These results clearly show that Vero cells grow with a satisfactory result while using the blood platelet lysate according to the present invention. The results obtained were fully comparable to those obtained with 5% FBS. However, the values of the blood platelet lysate according to the invention decreased on day 8, probably because the nutrient media were running out.

Table V shows the ratio of the cell concentration for selected days to the seeding concentration for Hybridoma 39.5 cells. The experiment was performed using bovine blood platelet lysate (BBPL), 5 different batches, and with FBS as a reference.

TABLE V

| | | 10% BBPL | 10% BBPL | 10% BBPL | 10% BBPL | 10% BBPL |
|---|---|---|---|---|---|---|
| Day | 5% FBS | (05) | (10) | (21) | (22) | (28) |
| 0 | 1.00 | 1.00 | 1.00 | 1.00 | 1.00 | 1.00 |
| 1 | 2.56 | 2.56 | 2.18 | 2.01 | 2.09 | 2.02 |
| 3 | 3.47 | 4.24 | 6.05 | 7.21 | 4.03 | 3.66 |
| 4 | 0.87 | 1.00 | 1.40 | 2.05 | 0.97 | 0.81 |

The results obtained in this experiment show that the Hybridoma 39.5 cells grow very satisfactorily while using the blood platelet lysate according to the present invention. The results obtained on days 3 and 4 after seeding were better than the result obtained with 5% FBS.

### Example 2b

### Cell Culture Experiment Using Porcine Blood Platelet Lysate

Table IV shows the ratio of the cell concentration for selected days to the seeding concentration for Vero cells. The experiment was performed with porcine blood platelet lysate (PBPL), 2 different concentrations, and with FBS and PS as references.

TABLE VI

| | 5% | 10% PBPL | 5% PBPL | | |
|---|---|---|---|---|---|
| Day | FBS | (03) | (03) | 10% PS | 5% PS |
| 0 | 1.00 | 1.00 | 1.00 | 1.00 | 1.00 |
| 1 | 0.63 | 0.73 | 0.65 | 0.44 | 0.46 |
| 2 | 5.08 | 5.98 | 4.67 | 3.80 | 2.56 |
| 4 | 12.84 | 12.05 | 9.90 | 8.13 | 7.23 |
| 7 | 24.36 | 17.93 | 11.48 | 13.08 | 9.58 |

These results clearly demonstrate that also the blood platelet lysate according to the invention, obtained from pig, gave satisfactory results in culture of Vero cells, which results were better than those obtained when using commercial PS.

Table VII shows the ratio of the cell concentration for selected days to the seeding concentration for 39.5 Hybridoma cells. The experiment was performed with porcine blood platelet lysate (PBPL), and with FBS and PS as references.

TABLE VII

| Day | 5% FBS | 10% PBPL | 5% PBPL | 10% PS | 5% PS |
|---|---|---|---|---|---|
| 0 | 1.00 | 1.00 | 1.00 | 1.00 | 1.00 |
| 1 | 2.67 | 2.32 | 1.84 | 2.72 | 2.47 |
| 2 | 10.88 | 13.27 | 8.16 | 11.07 | 9.29 |
| 3 | 16.51 | 15.82 | 9.29 | 17.30 | 10.45 |
| 4 | 6.46 | 6.97 | 7.56 | 9.40 | 4.36 |

These results clearly demonstrate that the blood platelet lysate according to the invention, obtained from pig, gave satisfactory results in culture of 39.5 Hybridoma cells.

Table VIII shows the ratio of the cell concentration for selected days to the seeding concentration for CHO cells. The experiment was performed with porcine blood platelet lysate (PBPL), 2 different concentrations, and with FBS and PS as references.

TABLE VIII

| | | 10% PBPL | 5% PBPL | | |
|---|---|---|---|---|---|
| Day | 5% FBS | (03) | (03) | 10% PS | 5% PS |
| 0 | 1.00 | 1.00 | 1.00 | 1.00 | 1.00 |
| 1 | 0.62 | 0.79 | 0.76 | 0.42 | 0.27 |
| 3 | 7.07 | 8.23 | 8.03 | 4.54 | 2.20 |
| 4 | 13.53 | 22.85 | 24.53 | 10.63 | 5.03 |
| 6 | 24.81 | 29.13 | 41.24 | 27.35 | 12.77 |

Also these results clearly demonstrate that use of porcine blood platelet lysate according to the invention gave very satisfactory results, in this case in culture of CHO cells, and constitutes an excellent substitute for both FBS and PS.

1. A method of preparing a blood platelet lysate starting from platelet-rich plasma from whole blood of animals, to which whole blood a citrate has been added to prevent coagulation,
comprising:
a) concentrating the platelet-rich plasma by ultra-filtration to obtain a concentrated platelet-rich plasma,
b) adding water to the concentrated platelet-rich plasma for lysis of the platelets included,
c) adding Ca²⁺ to the lysed concentrated platelet-rich plasma for forming coagel from other components than lysed platelets, the concentrated platelet-rich plasma, after addition of Ca²⁺, being allowed to stand at 37° C. maximum, for 3-8 h,
d) centrifuging the coagel, whereby a blood platelet lysate is obtained, which substantially consists of lysed platelets, and a coagulate, and
e) after centrifugation, the blood platelet lysate going through a filtering step.

2. A method as claimed in claim 1, wherein the protein content of the platelet-rich plasma in ultrafiltration is concentrated from about 6% to about 20%.

3. A method as claimed in claim 1, wherein the concentrated platelet-rich plasma after ultrafiltration is frozen for lysis and storage and/or analysis.

4. A method as claimed in claim 3, wherein after freezing the concentrated platelet-rich plasma is thawed.

5. A method as claimed in claim 4, wherein thawing of the concentrated platelet-rich plasma occurs at a temperature below 37° C.

6. A method as claimed in claim 5, wherein thawing occurs in a room with controlled temperature.

7. A method as claimed in claim 1, wherein the water added for lysis is deionised water.

8. A method as claimed in claim 1, wherein lysis by adding of water occurs at a temperature below 37° C.

9. A method as claimed in claim 8, wherein lysis occurs for at least 1 h.

10. A method as claimed in claim 1, wherein citrate is added to the lysate after centrifugation to prevent any post-coagulation.

11. A method as claimed in claim 10, wherein the amount of citrate added is 0.1-2.0 weight %.

12. A method as claimed in claim 1, wherein the platelet-rich plasma is of food quality.

13. A method as claimed in claim 1, wherein the filtering step comprises filtration by sterile filtration.

14. A method as claimed in claim 1, wherein the filtering step also comprises prefiltration.

15. A method as claimed in claim 14, wherein pre-filtering occurs through a filter cloth and/or prefilter.

16. A method as claimed in claim 1, wherein the platelet-rich plasma from whole blood of animals is from cattle or pig.

17. A method as claimed in claim 1, wherein the standing of step c) is carried for 5 h at 18-25° C.

18. A method as claimed in claim 1, wherein the protein content of the platelet-rich plasma in ultrafiltration is concentrated from about 6% to about 12%.

19. A method as claimed in claim 2, wherein the concentrated platelet-rich plasma after ultrafiltration is frozen for lysis and storage and/or analysis.

20. A method as claimed in claim 4, wherein thawing of the concentrated platelet-rich plasma occurs at a temperature below 20° C.

21. A method as claimed in claim 4, wherein thawing of the concentrated platelet-rich plasma occurs at a temperature below 12° C.

22. A method as claimed in claim 1, wherein lysis by adding of water occurs at a temperature below 22° C.

23. A method as claimed in claim 1, wherein lysis by adding of water occurs at a temperature below 5° C.

24. A method as claimed in claim 10, wherein the amount of citrate added is 0.3-1.0 weight %.
